(19) United States
(12) Patent Application Publication
(10) Pub. No.: US 2007/0017530 A1
(43) Pub. Date: Jan. 25, 2007

(54) DETECTING ELECTRICAL ACTIVITY AND ASSESSING AGENTS FOR THE ABILITY TO INFLUENCE ELECTRICAL ACTIVITY

(76) Inventors: Naweed I. Syed, Calgary (CA); Graham Arnold Jullien, Calgary (CA); Gerald Zamponi, Cochrane (CA)

Correspondence Address: FISH & RICHARDSON P.C., PO BOX 1022, MINNEAPOLIS, MN, 55440-1022, US

(21) Appl. No.: 11/423,380

(22) Filed: Jun. 9, 2006

Related U.S. Application Data
(60) Provisional application No. 60/689,645, filed on Jun. 10, 2005.

(51) Int. Cl. A61B 19/00

(52) U.S. Cl. 128/897

(57) ABSTRACT

This document provides assay systems related to detecting electrical activity and assessing agents for the ability to influence electrical activity. For example, methods and materials for identifying agents capable of treating seizure like behavior are provided herein.

### CROSS-REFERENCE TO RELATED APPLICATIONS

This application claims priority from U.S. Provisional Application Ser. No. 60/689,645, filed Jun. 10, 2005.

### BACKGROUND

1. Technical Field

This document relates to systems involved in detecting electrical activity and assessing agents for the ability to influence electrical activity. For example, this document relates to methods and materials that can be used to identify agents having the ability to reduce or prevent electrical activity (e.g., seizure like behavior in neurons).

2. Background Information

Seizure like behavior in neurons can be representative of several physiological and neurological conditions, such as epilepsy, which affect over one percent of the population. There can be many causes of this behavior. In practice, drugs are used to treat the symptoms of seizure like behavior in neurons.

High throughput drug screening assays are used to find compounds that are effective in reducing seizure like behavior. There, however, are limitations with these drugs and the screening assays used to discover them. First, many of the drugs that are used today to treat epilepsy are developed for specific protein targets. That is, the drugs are limited in their scope and only target specific proteins that are thought to contribute to seizure like behavior. This inherent limitation drastically constrains the identification of drugs that may act on other targets to reduce seizure like activity to help humans with epilepsy. Second, when measuring the effectiveness of a compound on network firing behavior of neurons, intracellular recordings are used. These recordings have very low throughput and do not allow for the screening of many compounds concurrently. Fluorescent, voltage sensitive dyes have also been used, but they result only in indirect readouts of network activity, and are often toxic to cells.

### SUMMARY

This document provides assay systems related to detecting electrical activity and assessing agents for the ability to influence electrical activity. For example, this document relates to methods and materials that can be used to identify agents capable of treating seizure like behavior. In some embodiments, this document provides devices and assay systems as well as methods for using such devices and assay systems to identify agents having the ability to reduce or prevent seizure like behavior in a mammal (e.g., a human). A seizure like behavior can be the seizures or convulsions associated with epilepsy. Such devices and assay systems can allow scientists, researchers, and drug developers to perform high throughput screens for agents effective in reducing or preventing seizure like behavior in neurons without focusing on a single potential drug target polypeptide. As described herein, electrical activity of neurons that is characteristic of seizure like behavior can be detected using integrated circuit technology and used to help determine whether or not a particular test compound is capable of inhibiting seizure like behavior. In some cases, the methods and materials provided herein can be used to identify an agent having the ability to reduce a seizure like behavior in a mammal while the mammal is experiencing a seizure like behavior, to reduce the incidence or severity of future seizure like behavior in a mammal (e.g. a mammal having experienced past seizure like behavior), or to prevent seizure like behavior from occurring in a mammal (e.g., a mammal having experienced past seizure like behavior).

In general, one aspect of this document features an assay system for testing the effects of test agents on neuronal networks that exhibit seizure like electrical activity. The assay system comprises, or consists essentially of, (a) an assay plate comprising a plurality of locations, wherein each of the plurality of locations defines a surface suitable for maintaining a mammalian neuronal cell network having the ability to create seizure like electrical activity, and wherein the assay plate is configured to retain a different test agent to each of the plurality of locations; (b) an electrical field sensing device for each of the plurality of locations, wherein each electrical field sensing device is configured to detect electrical activity from the mammalian neuronal cell network; and (c) a computer configured to process data obtained from each electrical field sensing device. The assay plate can comprise plastic. The assay plate can be a 96-well microtiter plate. Each of the plurality of locations can be a well in the assay plate. Each electrical field sensing device can be located within the well. Each electrical field sensing device can comprise a growth substrate coating. The growth substrate coating can be a poly-L-lysine coating, a lamine coating, a fibronectin coating, or a collegen coating. Each of the plurality of locations can comprise a chemical agent that promotes maintenance of the mammalian neuronal cell network on each electrical field sensing device. The chemical agent can be a neurotrophic factor or a growth factor. The chemical agent can be nerve growth factor or brain derive neurotrophic factor. The assay system can comprise a wired connection that connects each electrical field sensing device to the computer. The wired connection can be a direct wired connection connecting each electrical field sensing device to the computer. The assay system can comprise a wireless connection that connects each electrical field sensing device to the computer. The computer can comprise a processor and a connection to a power source. The assay system can comprise a controller that controls the voltage applied to each electrical field sensing device. The data can be sent to the computer via a wired connection. The data can be sent to the computer via a wireless connection. The computer can process the data using a software program. The software program can interpret the data and displays the amount of electrical activity from the mammalian neuronal cell network to a user. The assay plate can be configured to deliver a different test agent to each of the plurality of locations. The mammalian neuronal cell network can comprise cells selected from the group consisting of cortical neurons, hippocampal neurons, glutaminergic neurons, and glial cells. Each of the plurality of locations can comprise the mammalian neuronal cell network.

In another aspect, this document features a method for identifying an agent having the ability to inhibit seizure like electrical activity in a mammalian neuronal cell network. The method comprises, or consists essentially of, (a) providing an assay system comprising: (i) an assay plate comprising a plurality of locations, wherein each of the plurality of locations defines a surface comprising a mammalian neuronal cell network, and wherein the assay plate is configured to retain a different test agent to each of the plurality of locations, (ii) an electrical field sensing device for each of the plurality of locations, wherein each electrical field sensing device is configured to detect electrical activity from the mammalian neuronal cell network; and (iii) a computer configured to process data obtained from each electrical field sensing device; (b) adding a different test agent to each of the plurality of locations; and (c) determining whether or not the presence of a test agent in at least one of the plurality of locations inhibits seizure like electrical activity of the mammalian neuronal cell network, wherein inhibition of the seizure like electrical activity indicates that the test agent is the agent having the ability to inhibit seizure like electrical activity. The assay plate can comprise plastic. The assay plate can be a 96-well microtiter plate. Each of the plurality of locations can be a well in the assay plate. Each electrical field sensing device can be located within the well. Each electrical field sensing device can comprise a growth substrate coating. The growth substrate coating can be a poly-L-lysine coating, a lamine coating, a fibronectin coating, or a collegen coating. Each of the plurality of locations can comprise a chemical agent that promotes maintenance of the mammalian neuronal cell network on each electrical field sensing device. The chemical agent can be a neurotrophic factor or a growth factor. The chemical agent can be nerve growth factor or brain derive neurotrophic factor. The assay system can comprise a wired connection that connects each electrical field sensing device to the computer. The wired connection can be a direct wired connection connecting each electrical field sensing device to the computer. The assay system can comprise a wireless connection that connects each electrical field sensing device to the computer. The computer can comprise a processor and a connection to a power source. The assay system can comprise a controller that controls the voltage applied to each electrical field sensing device. The data can be sent to the computer via a wired connection. The data can be sent to the computer via a wireless connection. The computer can process the data using a software program. The software program can interpret the data and displays the amount of electrical activity from the mammalian neuronal cell network to a user. The assay plate can be configured to deliver a different test agent to each of the plurality of locations. The mammalian neuronal cell network can comprise cells selected from the group consisting of cortical neurons, hippocampal neurons, glutaminergic neurons, and glial cells. The mammalian neuronal cell network within each of the plurality of locations can be stimulated with an electrical pulse, a stimulating agent, or magnesium-free media before the adding step (b). The stimulating agent can comprise potassium. The stimulating agent can comprise glutamate. The stimulating agent can comprise cyclothiazide, coriaria lactone, or tutin. The electrical pulse can be provided by an electrode located within each of the plurality of locations.

The method can comprise determining the level of electrical activity at each of the plurality of locations (a) before stimulating the mammalian neuronal cell network within each of the plurality of locations and (b) before the adding step, thereby determining a normal activity index for each of the plurality of locations. The normal activity index for each of the plurality of locations can be stored via the computer. The method can comprise determining the level of seizure like electrical activity at each of the plurality of locations (a) after stimulating the mammalian neuronal cell network within each of the plurality of locations and (b) before the adding step, thereby determining an seizure activity index for each of the plurality of locations. The seizure activity index for each of the plurality of locations can be stored via the computer. The method can comprise determining the level of electrical activity at each of the plurality of locations (a) after stimulating the mammalian neuronal cell network within each of the plurality of locations and (b) after the adding step, thereby determining a test activity index for each of the plurality of locations. The test activity index for each of the plurality of locations can be stored via the computer. The determining step (c) can comprise comparing, for each of the plurality of locations, the test activity index to the normal activity index and the seizure activity index, wherein a test activity index having a level equal to the normal activity index or between the normal activity index and the seizure activity index indicates that the test agent that produced such a test activity index is the agent having the ability to inhibit seizure like electrical activity.

The method can comprise determining the level of seizure like electrical activity at each of the plurality of locations (a) after stimulating the mammalian neuronal cell network within each of the plurality of locations and (b) before the adding step, thereby determining an seizure activity index for each of the plurality of locations. The method can comprise determining the level of electrical activity at each of the plurality of locations (a) after stimulating the mammalian neuronal cell network within each of the plurality of locations and (b) after the adding step, thereby determining a test activity index for each of the plurality of locations. The determining step (c) can comprise comparing, for each of the plurality of locations, the test activity index to the seizure activity index, wherein a test activity index having a level less than the seizure activity index indicates that the test agent that produced such a test activity index is the agent having the ability to inhibit seizure like electrical activity.

The method can comprise using a pipette to place cells at each of the plurality of locations, thereby providing each of the plurality of locations with the mammalian neuronal cell network. The assay plate can be placed in an incubator to support growth or maintenance of the mammalian neuronal cell network. The computer can instruct each electrical field sensing device when to read electrical activity. The method can comprise, after step (c): (d) removing each different test agent from the plurality of locations; (e) adding a second set of test agents to the assay plate such that each of the plurality of locations comprises a different test agent from the second set; and (f) determining whether or not the presence of a test agent in at least one of the plurality of locations inhibits seizure like electrical activity of the mammalian neuronal cell network, wherein inhibition of the seizure like electrical activity indicates that the test agent is the agent having the ability to inhibit seizure like electrical activity. The method can comprise repeating steps (d) through (f) for a third set of test agents. The method can comprise repeating steps (d) through (f) for two to 1000 additional sets of test agents.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 includes a schematic cross-sectional view of a single well from an assay plate having multiple wells.

FIG. 2 is a flow diagram of steps that can be performed to identify agents having the ability to inhibit seizure like behavior.

FIG. 3 is a graph plotting the cellular activities under current clamp with the upward transitions reflecting action potentials that are being fired. The cells were pyramidal cells from the hippocampus at day 14 from a primary mouse CD1 neuronal culture. The cells were bathed in artificial cerebral spinal fluid (ACSF).

FIG. 4 is a graph plotting the cellular activities under current clamp with the upward transitions reflecting action potentials that are being fired. The cells were pyramidal cells from the hippocampus at day 14 from a primary mouse CD1 neuronal culture. The recording started when the external solution is switched to one with zero magnesium-ACSF (ZM-ACSF), and the seizure like behavior developed thereafter.

FIG. 5 is a schematic diagram of a computer that can be used with the assay systems provided herein as, for example, shown in FIG. 1.

### DETAILED DESCRIPTION

This document provides methods and materials related to detecting electrical activity and assessing agents for the ability to influence electrical activity. For example, this document relates to methods and materials that can be used to identify agents capable of treating seizure like behavior. In some embodiments, this document provides devices and assay systems as well as methods for using such devices and assay systems to identify agents having the ability to reduce or prevent seizure like behavior in a mammal (e.g., a human).

In some cases, an assay system provided herein can contain a substrate having multiple locations for growing or maintaining neuronal cell networks. The substrate can be any shape and can be made from any type of material (e.g., plastic, glass, or silicon). In one example, the substrate of an assay system provided herein can be an assay plate having multiple locations for growing or maintaining neuronal cell networks. The substrate (e.g., assay plate) can be configured such that each location is separated from each other location. In some cases, the substrate can be configured such that components (e.g., growth media, test agents, serum, growth factors, etc.) added to a particular location are retained in that location. For example, a standard 96-well tissue culture plate can be used as a substrate with each well being a location for growing or maintaining neuronal cell networks. In some cases, a substrate can be configured such that the multiple locations for growing or maintaining neuronal cell networks are wells, chambers, or cavities having at least one wall (e.g., one, two, three, four, or more walls). In some cases, the substrate can be configured to be flat with each location being separated from each other location via a chemical boundary. For example, the substrate can have a surface with hydrophobic and hydrophilic regions. The hydrophilic regions can be used as the location for growing or maintaining neuronal cell networks, while the hydrophobic regions can be used to retain the neuronal cell networks or components (e.g., growth media, test agents, serum, growth factors, etc.) to the hydrophobic regions. In some cases, the hydrophobic regions can be a series of vertical and horizontal lines, while the hydrophilic regions can be the square- or rectangle-shaped regions between those hydrophobic vertical and horizontal lines. Other shapes such as triangles, ovals, or circles can be used as well.

As described herein, each location can define, or can contain a structure (e.g., a chip) having, a surface that can be used as an attachment surface for growing or maintaining a neuronal cell network. Such an attachment surface can be any type of surface. For example, a surface for attachment of a neuronal cell network can be a silicon or germanium wafer or chip. A chip can be a slice of semiconducting material, such as silicon or germanium, doped and otherwise processed to have an electrical characteristic. In some cases, a chip can be an integrated circuit. Chips designed to contain electrical field sensing devices, electrodes, or both electrical field sensing devices and electrodes can be used in the devices and assay systems provided herein. In some cases, an attachment surface can be an integral part of the substrate. For example, the surface can be a silicon wafer that is configured to be the bottom of an assay plate. In some cases, an attachment surface can be a surface of structure that is added to a substrate already having a bottom. For example, an attachment surface can be the upper surface of a silicon wafer that is added to a standard 96-well tissue culture plate.

Each location of the substrate can contain an electrical field sensing device capable of detecting electrical currents of a neuronal cell network (e.g., a mammalian neuronal cell network). In some cases, the attachment surface of each location that is configured for neuronal cell network growth and maintenance can be a surface of an electrical field sensing device. Examples of electrical field sensing devices include, without limitation, field effect devices and sensitive voltage measuring instruments. Additional examples of electrical field sensing devices include, without limitation, those devices described elsewhere (Barbolina et al., “Submicron sensors of local electric field with single-electron resolution at room temperature,” Applied Physics Lett., 88(1): Art. No. 013901 (2006); Mamishev et al., “Interdigital Sensors and Transducers,” Proc. IEEE, 92(5):808-845 (2004); Shay and Zahn, “Cylindrincal Geometry Electroquasistatic Dielectrometry Sensors,” IEEE Conference on Electrical Insulation and Dielectric Phenomena, pp. 126-129 (2002); Preethichandra and Shida, “A Simple Interface Circuit to Measure Very Small Capacitance Changes in Capacitive Sensors,” IEEE Transactions on Instrumentation and Measurement, 50(6):1583-1586 (2001); Mamishev et al., “Assessment of Performance of Fringing Electric Field Sensor Arrays,” IEEE Conference on Electrical Insulation and Dielectric Phenomena, pp. 918-921 (2002); and Khan and Abdullah, “Finite-Element Modeling of Multielectrode Capacitive Systems for Flow Imaging,” IEE Proceedings-G Circuits Devices and Systems, 140(3):216-222 (1993)).

In some embodiments, the electrical field sensing devices of a substrate can form a transistor sensing array or transistor array, and a microcontroller can be used and configured to be contained in a single integrated circuit or transistor chip. A transistor array can be a linear or two-dimensional array of sensors that respond to absolute or incremental direct changes in electric fields. In some cases, an electrical field sensing device can be a sensing transistor or a field effect transistor that changes the conductivity of a conducting channel as a function of an electric field established across a thin oxide (dielectric) region above the channel. In one embodiment, each well of a 96-well tissue culture plate can have a chip containing one or more electrical field sensing devices. In this case, a neuronal cell network can be grown or placed onto an upper surface of the chip located in each well.

The devices and assay systems provided herein can be configured to use components and techniques such as field-effect transistors, high impedance voltage meters, monitoring changes in spacing of charged electrodes, optical or electron beam deflection, capacitance change sensors, Kelvin probes, and other forms of charge sensing electrodes to detect electrical activity of a neuronal cell network.

Each location of the substrate can contain an electrode capable of stimulating a neuronal cell network. For example, each location of the substrate can contain an electrode capable delivering voltage pulses up to 7 volts (e.g., 1, 2, 3, 4, 5, 6, or 7 volts) amplitude to a neuronal cell network. In some cases, the surface of each location that is configured for neuronal cell network growth and maintenance can be a surface of an electrode. Examples of electrodes include, without limitation, aluminum, gold, titanium, platinum, tungsten, and polysilicon electrodes.

In some cases, the surface configured for neuronal cell network growth and maintenance can function as both electrical field sensing device and electrode. For example, a chip can be designed to provide a surface for neuronal cell network growth or maintenance where the chip contains can electrical field sensing device component and electrode component. An electrode can be combined with an electrical field sensing device using a 3-D sandwich of the DALSA HV CMOS technology and the TSMC 180 nm CMOS technology. The DALSA technology can allow the use of voltages greater than 3 volts for stimulation, while the TSMC technology can provide sensitive electrical field sensing. The TSMC (Taiwan Semiconductor Manufacturing Corporation) 180 nm process is a standard CMOS fabrication process available for the fabrication of chips through a multiproject wafer system (see, e.g., TSMC's internet site at “tsmc.com/english/technology/t0103” dot “htm”). In some cases, an on-chip processor can be used to correct for sensor variation similar to the approaches used for CMOS or CCD imagers.

A neuronal cell network can include any type of neuron. For example, a neuronal cell network can have cortical neurons, hippocampal neurons, glutaminergic neurons, or combinations thereof. In some cases, a neuronal cell network can be a brain slice (e.g., a hippocampal slice). A neuronal cell network can be formed using a primary neuronal culture or a neuronal cell line culture. In some cases, a neuronal cell network can contain non-neuronal cells. For example, a neuronal cell network can contain neurons and glial cells (e.g., oligodendrocytes, astrocytes, or microglial cells). In some cases, a mammalian neuronal cell network can be used in a device, assay system, or method provided herein. For example, a neuronal cell network containing human, monkey, dog, cat, horse, cow, sheep, rat, or mouse neurons can be used.

Any method can be used to obtain neurons and other cells (e.g., glial cells). For example, standard tissue biopsy and tissue culture techniques can be used to obtain primary neuronal cell cultures. In some cases, neuronal tissue (e.g., brain tissue) can be obtained, disassociated, and cultured in a tissue culture dish pre-treated with serum. Once non-neuronal cells adhere to the surface, the neuronal cells can be collected using, for example, a pipette. For example, neuronal somata can be selectively drawn into a fire-polished, glass pipette that is pre-treated with serum and is attached to a syringe. This can be performed using a pseudo-dark field illuminator. Gentle suction pressure can be applied to remove neuronal cell bodies from the culture. Once collected, the neuronal cells can be applied to the surfaces at each location of the substrate (e.g., assay plate). The surfaces at each location can contain a growth substrate coating to promote neuronal cell attachment, neuronal cell growth, and/or the maintenance of neuronal cell survival. Examples of growth substrate coatings include, without limitation, poly-L-lysine coatings, lamine coatings, fibronectin coatings, or collegen coatings. Once the neuronal cells attach to a surface of a substrate (e.g., a poly-L-lysine coated surface of a substrate), the cells remaining in the tissue culture dish after the neuronal cells were collected can be added to the neuronal cells. Any amount of time can be used to allow neuronal cells to attach to a surface. For example, neuronal cells can attach in 15, 20, 30, 45, 60, or more minutes. In some cases, a neuronal cell network can be obtained as described elsewhere (Khosravani et al., FEBS Letters, 579(29):6587-94 (2005)).

The devices and assay systems provided herein can be configured such that a substrate has multiple locations each of which contains an electrical field sensing device designed to sense electrical activity from a neuronal cell network. In general, the neuronal cell network is located on top of the electric field sensing device so that the device can detect changes in neuronal firing behavior. In some cases, the electrical field sensing device is able to read the electrical firing patterns of individual cells or groups of cells (e.g., tens to hundreds of cells such as the neurons of a neuronal cell network). The measurements of electrical activity produced by a neuronal cell network can be taken at predetermined intervals or continuously.

The electrical field sensing devices at each location of the substrate (e.g., wells of an assay plate) can be interfaced with a software-controlled computer system. The interface can be either a wired or wireless connection. Pre-processing on the electrical field sensing devices themselves may also occur. Such pre-processing includes, without limitation, data extraction, signal conditioning, and filtering. The computer can read where and how much electrical activity is detected at the different locations on the substrate. This reading can be done in real-time to monitor electrical cellular activity.

In some cases, the devices and assay systems provided herein can include a data output microprocessor to stream real time data to a computer. Analog outputs can be converted to digital values inside a microcontroller using analog to digital (AD) converters.

The devices and assay systems provided herein can be used to identify test agents having the ability to inhibit seizure like behavior. For example, each electrical field sensing device within each location can be used to measure the normal electrical activity produced by a neuronal cell network. This normal electrical activity can be the activity produced by neuronal cell network in the absence of the test agent to be tested. In some cases, a normal electrical activity can be the activity detected from the neuronal cell network in the absence of external neuronal stimulation such as electrical current from an electrode or a chemical treatment (e.g., exposure to increased potassium levels). In some cases, the level of normal electrical activity measured from a particular neuronal cell network can be used to calculate a normal activity index for that particular neuronal cell network. In one example, the level of normal electrical activity measured for a particular location can be used as a normal activity index for that location. In some cases, a normal activity index can be adjusted to account for variability based on measurements obtained from other neuronal cell networks present on the same substrate or based on repeated measurements from the same neuronal cell network. For example, the level of normal electrical activity can be measured for all the locations of a particular substrate. The measured normal electrical activity levels then can be used to determine a single normal activity index based on all the measurements. For example, a normal activity index can be the average level of normal electrical activity based on measurements from all the locations on a substrate. In another example, a normal activity index can be the average level of normal electrical activity based on multiple measurements obtained from the same location on a substrate.

Any method can be used to measure electrical activity from a neuronal cell network. For example, electrical activity can be determined using an electrical field sensing device to measure the number and magnitude of electrical discharges (e.g., spontaneous electrical discharges) that occur in the functional neuronal cell network during a predefined time period (e.g., in the milliseconds to minutes range). In some cases, the predefined time period can be 1 millisecond, 2 milliseconds, 3 milliseconds, 4 milliseconds, 5 milliseconds, 1 second, 2 seconds, 3 seconds, 4 seconds, 5 seconds, 15 seconds, 30 seconds, 45 seconds, 1 minute, 2, minutes, 3 minutes, 5 minutes, or more).

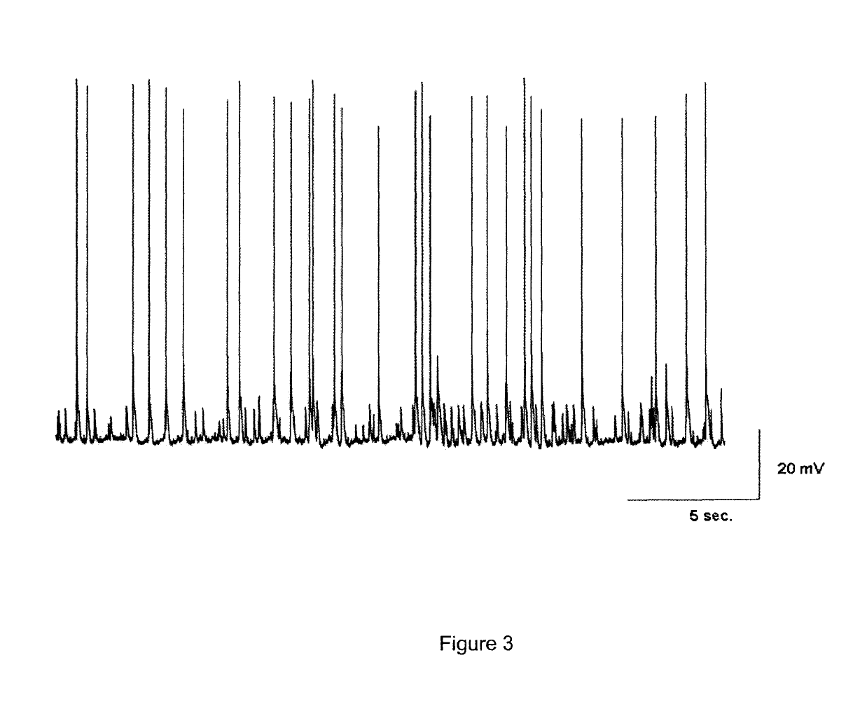

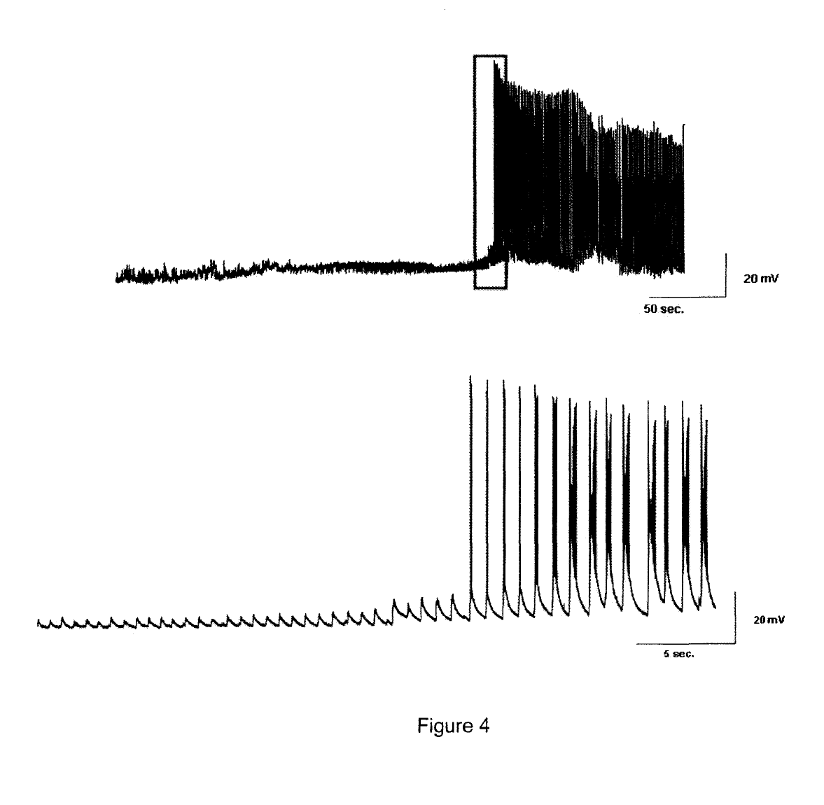

After measuring a normal electrical activity or determining a normal activity index for a particular neuronal cell network, the neuronal cell network can be artificially stimulated to exhibit seizure like behavior. Any method can be used to stimulate a seizure like behavior in a neuronal cell network. For example, electrical currents provided from electrodes present at each location of a substrate can be used stimulate the neuronal cell networks at each location. In some cases, application of high concentrations of potassium, or treatment with glutamate, cyclothiazide, coriaria lactone, tutin, or GABA receptor antagonists can be used to stimulate a neuronal cell network. In some cases, exposing a neuronal cell network to little or no magnesium can be used to stimulate a neuronal cell network. These treatments can result in different types of seizure like behavior in neuronal cell networks. An external influence on a neuronal cell network can lead to electrical spike and waveform discharges that are similar to human absence seizure activity. An electrical spike and waveform discharge can be a neuronal firing pattern that consists of a spike followed by a slow wave. An example of cells exhibiting normal electrical activity is set forth in FIG. 3, while an example of cells exhibiting a seizure like behavior is set forth in FIG. 4.

The process for stimulating a neuronal cell network can be automated and can include using, for example, a liquid handling robotic device. For example, a robotic pipetting system can be used to deliver a stimulatory dose of potassium or glutamate to all the locations (e.g., wells) of a substrate (e.g., 96-well assay plate). Such devices can be used to stimulate neuronal cell networks automatically in many locations (e.g., wells) quickly and reproducibly.

The electrical field sensing devices can be used to measure the activity levels of the stimulated neuronal cell networks, for example, over time. Such measurements can be used to determine a level of stimulated electrical activity for each particular neuronal cell network. For example, a stimulated electrical activity can be the activity produced by a neuronal cell network artificially stimulated. In some cases, the level of stimulated electrical activity measured from a particular neuronal cell network can be used to calculate a seizure activity index for that particular neuronal cell network. In one example, the level of stimulated electrical activity measured for a particular location can be used as a seizure activity index for that location. In some cases, a seizure activity index can be adjusted to account for variability based on measurements obtained from other neuronal cell networks present on the same substrate or based on repeated measurements from the same neuronal cell network. For example, the level of stimulated electrical activity can be measured for all the locations of a particular substrate. The measured level of stimulated electrical activity then can be used to determine a single seizure activity index based on all the measurements. For example, a seizure activity index can be the average level of stimulated electrical activity based on measurements from all the locations on a substrate. In another example, a seizure activity index can be the average level of stimulated electrical activity based on multiple measurements obtained from the same location on a substrate. In some cases, an increased network activity, as compared to the determined level of normal electrical activity or the normal activity index for a particular neuronal cell network, can be used to determine a seizure activity index.

The temporal and spatial distribution of neuronal activity within a neuronal cell network can be multiplexed into the data stream as it is acquired from the electrical field sensing devices, thus providing information about the synchrony within the electrical activities produced by the neuronal cell network. The electrical field sensing devices can be used to determine other information including, without limitation, the number of spikes per burst, the total number of bursts, or the interburst interval.

After measuring the level of stimulated electrical activity or determining a seizure activity index for a particular neuronal cell network, the neuronal cell network can be exposed to a particular test agent, exposed to conditions capable of artificially stimulating a neuronal cell network as described herein, and assessed for electrical activity to determine whether or not the test agent inhibits seizure like behavior. As used herein, the term “test agent” includes any agent that is suspected to have a biological effect such as action in a cell. A test agent can be a small organic molecule having, for example, 50 or fewer non-hydrogen atoms, or a polypeptide (e.g., an antibody), a nucleotide, an amino acid, a nucleic acid molecule, or a polysaccharide. In some cases, a test agent can be a polypeptide having a molecular weight less than 5,000 Daltons.

As described herein, any method can be used to stimulate a neuronal cell network to exhibit a seizure like behavior. In some cases, the same method used to stimulate a neuronal cell network for determining a level of stimulated electrical activity or a seizure activity index can be used to stimulate a neuronal cell network exposed to a test agent. For example, removal of magnesium from culture media can be used to stimulate a neuronal cell network at a particular location both for determining a seizure activity index and for determining whether or not the presence of a particular test agent inhibits seizure like behavior.

In some cases, determining whether or not the presence of a particular test agent inhibits seizure like behavior includes (1) determining a level of test electrical activity or a test activity index for a particular location exposed to a particular test agent under conditions of artificial stimulation and (2) comparing that level of test electrical activity or test activity index to a level of stimulated electrical activity, a seizure activity index, a level of normal electrical activity, a normal activity index, or a combination thereof. In general, the closer the level of test electrical activity or the test activity index is to the level of normal electrical activity or the normal activity index for a particular location, the more effective the test agent can be in reducing seizure like behavior. If the level of test electrical activity or the test activity index is less than the level of stimulated electrical activity or the seizure activity index for that particular location, then the test agent added to that location can be capable of inhibiting seizure like behavior. Likewise, if the level of test electrical activity or the test activity index is similar to the level of normal electrical activity or the normal activity index for that particular location, then the test agent added to that location can be capable of inhibiting seizure like behavior. If the level of test electrical activity or the test activity index is less than the level of normal electrical activity or the normal activity index for that particular location, then the test agent added to that location can be capable of inhibiting seizure like behavior. In this case, the test agent can be tested for non-specific and toxic effects on the neuronal cell network. If the test agent is determined to inhibit seizure like behavior in an undesired, non-specific manner such as via neuronal cell toxicity, then the test agent can be potentially removed from further consideration. In some cases, a desired test agent can be an agent having the ability to remove aberrant synchrony within a neuronal cell network in addition to having the ability to maintain normal electrical activity (e.g., normal intrinsic activity).

The electrical field sensing devices of the devices and assay systems provided herein can be used to measure a test electrical activity. A test electrical activity can be the activity produced by a neuronal cell network that is in the presence of a test agent and is under conditions capable of inducing stimulation. In some cases, the level of test electrical activity measured from a particular neuronal cell network can be used to calculate a test activity index for that particular neuronal cell network. In one example, the level of test electrical activity measured for a particular location can be used as a test activity index for that location. In some cases, a test activity index can be adjusted to account for variability based on repeated measurements from the same neuronal cell network. For example, a test activity index can be the average level of test electrical activity based on multiple measurements obtained from the same location on a substrate.

Any method can be used to contact a neuronal cell network with a test agent. For example, the process for contacting a neuronal cell network with a test agent can be automated and can include using, for example, a liquid handling robotic device. In some cases, a robotic pipetting system can be used to deliver a predetermined dose (e.g., 100 nM, 1 μM, 5 μM, 10 μM, 50 μM, 100 μM, or more) of a test agent to each location such that each location (e.g., each well) of a substrate (e.g., 96-well assay plate) receives a different test agent. Such devices can be used to deliver test agents to neuronal cell networks automatically in a quick and reproducible manner. In some cases, test agents can be delivered to each location via perfusion or microperfusion. For example, a test agent can be applied to a neuronal cell network by replacing the solution normally surrounding the cells with a solution containing the test agent to be applied.

After assessing a test agent, the neuronal cell network can be artificially stimulated as described herein (e.g., via potassium chloride depolarization) to ensure the ability of the neuronal cell network to fire at the end of the screen. For example, after testing 96 different test agents, each neuronal cell network of a 96-well plate can be stimulated via treatment with potassium in the absence of test agents to confirm that each neuronal cell network retained the ability to produce a seizure like behavior. Such a confirmation can be performed at other times as well. For example, each neuronal cell network can be stimulated to confirm each neuronal cell network's ability to produce a seizure like behavior before measuring normal electrical activity or determining a normal activity index. In such cases, the stimulatory treatment can be removed, and the neuronal cell networks can be allowed to establish normal activity patterns before measuring the normal electrical activity or determining a normal activity index.

A software system can be used to calculate a level of normal electrical activity, a normal activity index, a level of stimulated electrical activity, a seizure activity index, a level of test electrical activity, or a test activity index. These levels and indexes can be represented by numerical values that correspond to the average frequency and amplitudes of cellular firing events within a predefined period (e.g., milliseconds, seconds, or minutes). In some cases, the predefined period can be 1 millisecond, 2 milliseconds, 3 milliseconds, 4 milliseconds, 5 milliseconds, 1 second, 2 seconds, 3 seconds, 4 seconds, 5 seconds, 15 seconds, 30 seconds, 45 seconds, 1 minute, 2, minutes, 3 minutes, 5 minutes, or more). In some cases, additional characteristics of firing behavior and synchrony of firing can be used. The software system can use the collected information to generate statistical reports on the effect of a test agent being tested.

The methods, devices, and assay systems provided herein can be used to perform high-throughput screens to agents having the ability to inhibit seizure like behavior. In some cases, the methods and materials provided herein can be used to remove or reduce experimental variability that can be inherent when using different neuronal cell networks as two neuronal cell networks will never act the same. For example, measuring the before and after state of a test agent's impact on a single neuronal cell network and then repeating that measurement many times with many different individual groups of neurons can remove or minimize any issue of variability. By measuring this information on a per neuronal cell network basis, the software can statically analyzes which test agents are more reliable in generally reducing seizure like behavior.

As described herein, the effects of a test agent can be directly measured, instead of indirectly such as through the use of fluorescent dyes, as the electrical field sensing devices can measure electrical activity directly across an entire culture. This can result in a much simpler and more reliable data analysis to understand how test agents affect cellular physiology under normal and various pathological conditions. In addition, this can reduce or eliminate the perturbation of the native intracellular environment of the neurons and can reduce or eliminate cellular toxicity.

In addition to being used to identify test agents having the ability to inhibit seizure like behavior, the devices and assay systems provided herein can be used to evaluate neuronal cell network properties after long term exposure to epileptiform activity or can be used to investigate the firing properties of neuronal cell networks in general.

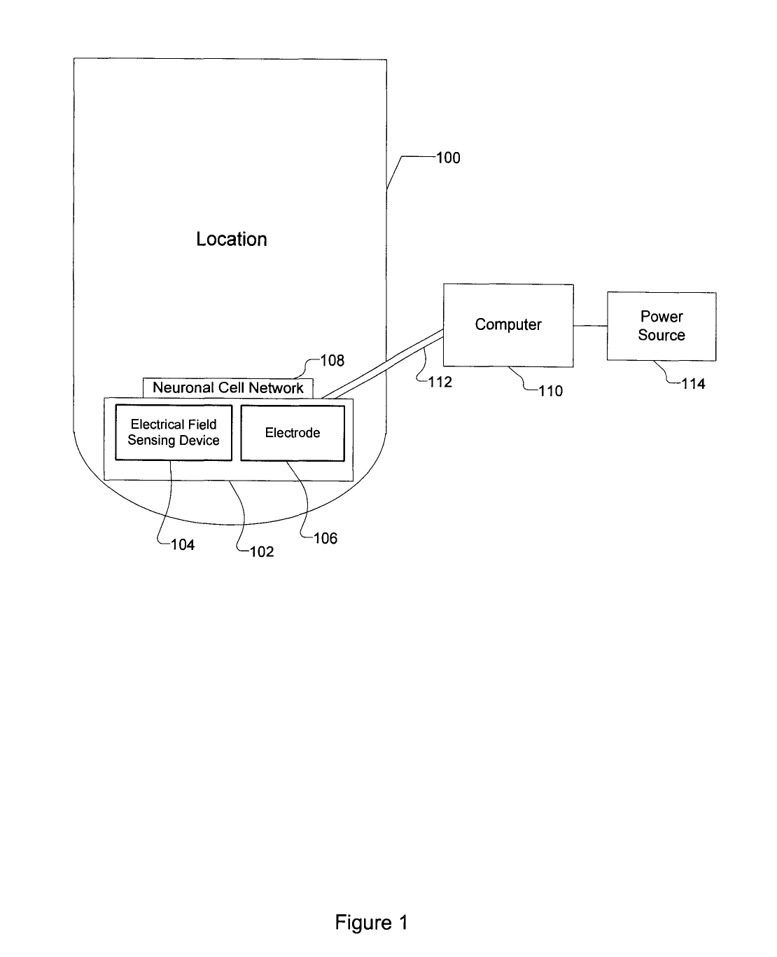

As described herein, a substrate can have multiple locations with each location having an electrical field sensing device defining a surface capable of supporting a neuronal cell network. In some cases, such a substrate can be an assay plate having a plurality of wells with each well having an electrical field sensing device. With reference to FIG. 1, an assay plate can contain a plurality of locations such as location 100. Location 100 can be any size (e.g., a standard well size from a 96-well tissue culture plate) and any shape (e.g., a rounded well). Location 100 can have a rounded bottom. In some cases, the bottom of a location can be flat. Location 100 can contain chip 102. Chip 102 can be designed to have electrical field sensing device 104, electrode 106, or both. Chip 102 can be any size and can be any shape. Typically, chip 102 is designed to fit into a standard well from a 96-well tissue culture plate. In some cases, chip 102 can be designed to form the bottom of location 100. Electrical field sensing device 104 can be designed to detect electrical activity from neuronal cell network 108 grown on a surface of chip 102. Neuronal cell network 108 can be a network on any type of neuron (e.g., a mammalian neuronal cell network). Electrode 106 can be designed to deliver electrical current to neuronal cell network 108 grown on a surface of chip 102. Computer 110 can be used to control the functions of electrical field sensing device 104 and electrode 106 of chip 102. For example, computer 110 can contain software instructions that direct electrical field sensing device 104 to measure electrical activity from neuronal cell network 108 at predetermined times for predetermined durations. Computer 110 can collect and process information received from electrical field sensing device 104 and electrode 106 of chip 102. Connection 112 can allow computer 110 to communicate with electrical field sensing device 104 and electrode 106 of chip 102. Connection 112 can be a wired connection or a wireless connection. Connection 112 can be bidirectional such that computer 110 can send signals to electrical field sensing device 104 of chip 102, electrode 106 of chip 102, or both and such that electrical field sensing device 104 of chip 102, electrode 106 of chip 102, or both can send signals to computer 110. Power supply 114 can be designed to provide a power source to computer 110, electrical field sensing device 104 of chip 102, electrode 106 of chip 102, or combinations thereof.

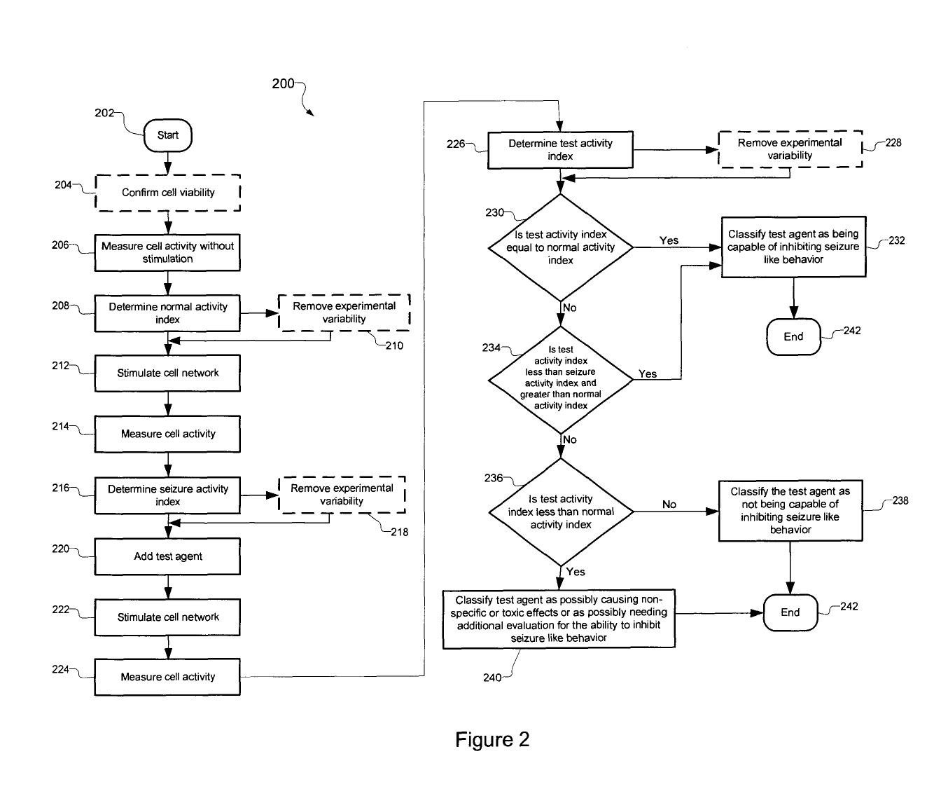

In some cases, the acts set forth in FIG. 2 can be performed to identify a test agent having the ability to inhibit seizure like behavior using the devices and assay systems provided herein. Procedure 200 can start with box 202. Box 204 represents optional acts that can be performed to confirm viability of a neuronal cell network. Such acts can include confirming the ability of a neuronal cell network to exhibit a seizure like behavior. Box 206 represents acts that can be performed to measure cell activity without artificial stimulation. For example, an electrical field sensing device can be instructed to measure the activity from a neuronal cell network under conditions that do not produce a seizure like behavior (e.g., normal tissue culture conditions). Box 208 represents acts that can be performed to determine a normal activity index. For example, the measurements obtained performing the acts of box 206 can be used to calculate a normal activity index. Box 210 represents optional acts that can be performed to reduce or remove experimental variability. Such acts can include making multiple measurements from a single neuronal cell network or multiple different neuronal cell networks.

Box 212 represents acts that can be performed to stimulate a neuronal cell network. For example, an electrode can be instructed to deliver electrical current to a neuronal cell network or a robotic device can be instructed to deliver one or more agents capable of stimulating a neuronal cell network. Box 214 represents acts that can be performed to measure cell activity. For example, an electrical field sensing device can be instructed to measure the activity from a neuronal cell network under conditions that produce a seizure like behavior. Box 216 represents acts that can be performed to determine a seizure activity index. For example, the measurements obtained performing the acts of box 214 can be used to calculate a seizure activity index. Box 218 represents optional acts that can be performed to reduce or remove experimental variability. Such acts can include making multiple measurements from a single neuronal cell network or multiple different neuronal cell networks under conditions capable of stimulating a neuronal cell network to exhibit seizure like behavior.

Box 220 represents acts that can be performed to add a test agent to a neuronal cell network. For example, a robotic device can be instructed to deliver a different test agent to each location of an assay plate. Box 222 represents acts that can be performed to stimulate a neuronal cell network. For example, an electrode can be instructed to deliver electrical current to a neuronal cell network or a robotic device can be instructed to deliver one or more agents capable of stimulating a neuronal cell network. In some cases, the acts of box 222 can be the same as the acts of box 212. Box 224 represents acts that can be performed to measure cell activity. For example, an electrical field sensing device can be instructed to measure the activity from a neuronal cell network exposed to a test agent and under conditions that are capable of producing a seizure like behavior in a neuronal cell network not exposed to a test agent. Box 226 represents acts that can be performed to determine a test activity index. For example, the measurements obtained performing the acts of box 224 can be used to calculate a test activity index. Box 228 represents optional acts that can be performed to reduce or remove experimental variability. Such acts can include making multiple measurements from the same neuronal cell network.

Boxes 230, 234, and 236 represent acts that can be performed to determine whether or not a test agent is capable of inhibiting seizure like behavior. For example, box 230 includes determining whether the test activity index is equal to the normal activity index. If yes for box 230, then the test agent can be classified as being capable of inhibiting seizure like behavior (box 232). If no for box 230, then the acts represented by box 234 can be performed. Box 234 includes determining whether the test activity index is less than the seizure activity index and greater than the normal activity index. If yes for box 234, then the test agent can be classified as being capable of inhibiting seizure like behavior (box 232).

If no for box 234, then the acts represented by box 236 can be performed. Box 236 includes determining whether the test activity index is less than the normal activity index. If no for box 236, then the test agent can be classified as not being capable of inhibiting seizure like behavior (box 238). If yes for box 236, then the test agent can be classified as possibly causing non-specific or toxic effects or as possibly needing additional evaluation for the ability to inhibit seizure like behavior (box 240). After classifying a test agent as set forth in box 232, 238, or 240, procedure 200 can stop with box 242.

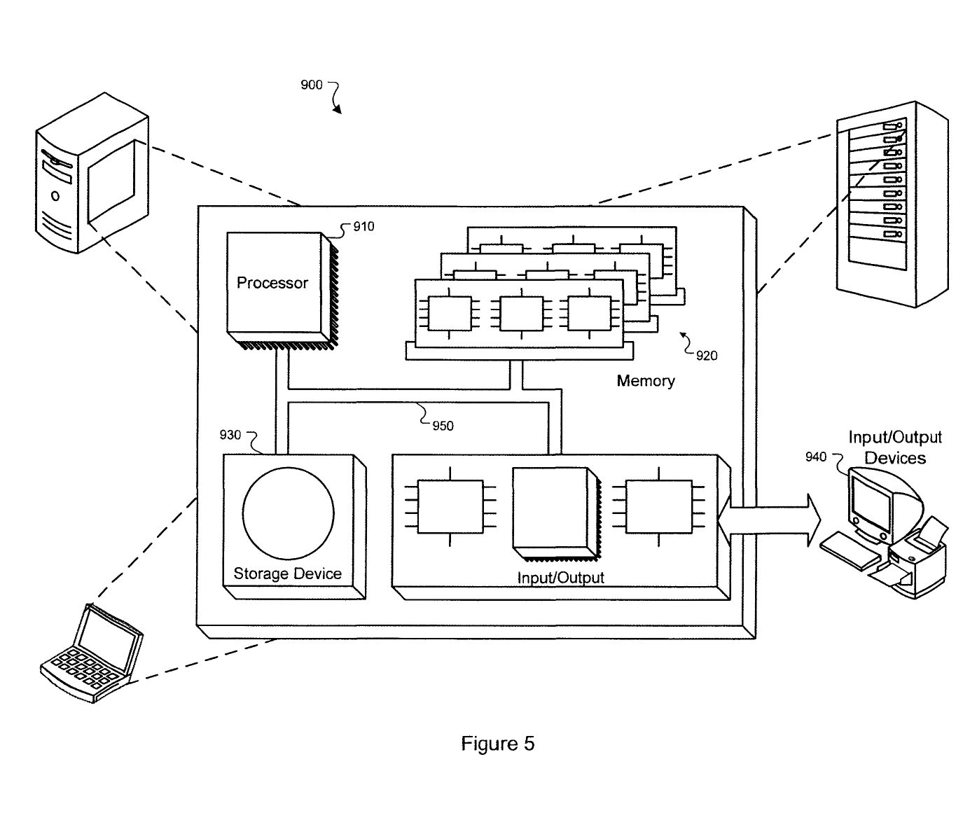

FIG. 5 is a schematic diagram of a general computer system 900. The system 900 includes a processor 910, a memory 920, a storage device 930, and an input/output device 940. Each of the components 910, 920, 930, and 940 are interconnected using a system bus 990. The processor 910 is capable of processing instructions for execution within the system 900. For example, the processor 910 can be a microcontroller that executes instructions that carry out the procedures described herein (e.g., procedure 200 set forth in FIG. 2). In some implementations, the processor 910 is a single-threaded processor. In other implementations, the processor 910 is a multi-threaded processor. The processor 910 is capable of processing instructions stored in the memory 920 or on the storage device 930. In some implementations, the processed instructions may generate graphical information for a user interface, on the input/output device 940.

The memory 920, which is a computer-readable medium, stores information within the system 900. In some implementations, the memory 920 is a volatile memory unit. In other implementations, the memory 920 is a non-volatile memory unit. The memory may be suitable for tangibly embodying computer program instructions and data. The instructions and data can be loaded into memory from an external source, such as the storage device 930 or the input/output device 940.

The storage device 930 is capable of providing mass storage for the system 900. In some implementations, the storage device 930 is a computer-readable medium. In various different implementations, the storage device 930 may be a floppy disk device, a hard disk device, an optical disk device, or a tape device.

The input/output device 940 provides input/output operations for the system 900. In some implementations, the input/output device 940 includes a keyboard and/or pointing device. In other implementations, the input/output device 940 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device or in a propagated signal, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The components of the system can be connected by any form or medium of digital data communication such as a communication network. Examples of communication networks include, e.g., a LAN, a WAN, and the computers and networks forming the Internet.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

The invention will be further described in the following examples, which do not limit the scope of the invention described in the claims.

### EXAMPLES

### Example 1

### High-throughput Screen for Agents having Anti-Epileptic or Anti-Convulsion Activity

Hippocampal neurons are added to each well of a 96-well plate having a chip located in each well. Each chip contains an electrical field sensing device and is connected to a computer. The hippocampal neurons are allowed to grow and form a neuronal cell network on a surface of each chip. The computer is used to direct the measurement and recording of electrical activity in each well to obtain a normal activity index for each neuronal cell network. A robotic device is used to replace the media in each well with media lacking magnesium. Once the neuronal cell networks are stimulated to exhibit seizure like behavior, the computer is used to direct the measurement and recording of electrical activity in each well to obtain a seizure activity index for each neuronal cell network.

After a robotic device is used to add a different test agent to each well and each neuronal cell network is stimulated, the computer is used to direct the measurement and recording of electrical activity in each well to obtain a test activity index for each neuronal cell network. For each well, the computer then compares the test activity index with the normal activity index and seizure activity index to determine whether or not the test agent is capable of inhibiting seizure like behavior. The results are displayed to a user on a computer monitor or in print form.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An assay system for testing the effects of test agents on neuronal networks that exhibit seizure like electrical activity, wherein said assay system comprises:
(a) an assay plate comprising a plurality of locations, wherein each of said plurality of locations defines a surface suitable for maintaining a mammalian neuronal cell network having the ability to create seizure like electrical activity, and wherein said assay plate is configured to retain a different test agent to each of said plurality of locations;
(b) an electrical field sensing device for each of said plurality of locations, wherein each electrical field sensing device is configured to detect electrical activity from said mammalian neuronal cell network; and
(c) a computer configured to process data obtained from each electrical field sensing device.

2. The assay system of claim 1, wherein said assay plate comprises plastic.

3. The assay system of claim 1, wherein said assay plate comprises a 96-well microtiter plate.

4. The assay system of claim 1, wherein each of said plurality of locations is a well in said assay plate.

5. The assay system of claim 4, wherein each electrical field sensing device is located within said well.

6. The assay system of claim 1, wherein each electrical field sensing device comprises a growth substrate coating.

7. The assay system of claim 6, wherein said growth substrate coating is a poly-L-lysine coating, a lamine coating, a fibronectin coating, or a collegen coating.

8. The assay system of claim 1, wherein each of said plurality of locations comprises a chemical agent that promotes maintenance of said mammalian neuronal cell network on each electrical field sensing device.

9. The assay system of claim 8, wherein said chemical agent is a neurotrophic factor or a growth factor.

10. The assay system of claim 8, wherein said chemical agent is nerve growth factor or brain derive neurotrophic factor.

11. The assay system of claim 1, wherein said assay system comprises a wired connection that connects each electrical field sensing device to said computer.

12. The assay system of claim 11, wherein said wired connection is a direct wired connection connecting each electrical field sensing device to said computer.

13. The assay system of claim 1, wherein said assay system comprises a wireless connection that connects each electrical field sensing device to said computer.

14. The assay system of claim 1, wherein said computer comprises a processor and a connection to a power source.

15. The assay system of claim 1, wherein said assay system comprises a controller that controls the voltage applied to each electrical field sensing device.

16. The assay system of claim 1, wherein said data is sent to said computer via a wired connection.

17. The assay system of claim 1, wherein said data is sent to said computer via a wireless connection.

18. The assay system of claim 1, wherein said computer processes said data using a software program.

19. The assay system of claim 18, wherein said software program interprets said data and displays the amount of electrical activity from said mammalian neuronal cell network to a user.

20. The assay system of claim 1, wherein said assay plate is configured to deliver a different test agent to each of said plurality of locations.

21. The assay system of claim 1, wherein said mammalian neuronal cell network comprises cells selected from the group consisting of cortical neurons, hippocampal neurons, glutaminergic neurons, and glial cells.

22. The assay system of claim 1, wherein each of said plurality of locations comprises said mammalian neuronal cell network.

23. A method for identifying an agent having the ability to inhibit seizure like electrical activity in a mammalian neuronal cell network, said method comprising:
(a) providing an assay system comprising:
(i) an assay plate comprising a plurality of locations, wherein each of said plurality of locations defines a surface comprising a mammalian neuronal cell network, and wherein said assay plate is configured to retain a different test agent to each of said plurality of locations,
(ii) an electrical field sensing device for each of said plurality of locations, wherein each electrical field sensing device is configured to detect electrical activity from said mammalian neuronal cell network; and
(iii) a computer configured to process data obtained from each electrical field sensing device;
(b) adding a different test agent to each of said plurality of locations; and
(c) determining whether or not the presence of a test agent in at least one of said plurality of locations inhibits seizure like electrical activity of said mammalian neuronal cell network, wherein inhibition of said seizure like electrical activity indicates that said test agent is said agent having the ability to inhibit seizure like electrical activity.

24. The method of claim 23, wherein said assay plate comprises plastic.

25. The method of claim 23, wherein said assay plate comprises a 96-well microtiter plate.

26. The method of claim 23, wherein each of said plurality of locations is a well in said assay plate.

27. The method of claim 26, wherein each electrical field sensing device is located within said well.

28. The method of claim 23, wherein each electrical field sensing device comprises a growth substrate coating.

29. The method of claim 28, wherein said growth substrate coating is a poly-L-lysine coating, a lamine coating, a fibronectin coating, or a collegen coating.

30. The method of claim 23, wherein each of said plurality of locations comprises a chemical agent that promotes maintenance of said mammalian neuronal cell network on each electrical field sensing device.

31. The method of claim 30, wherein said chemical agent is a neurotrophic factor or a growth factor.

32. The method of claim 30, wherein said chemical agent is nerve growth factor or brain derive neurotrophic factor.

33. The method of claim 23, wherein said assay system comprises a wired connection that connects each electrical field sensing device to said computer.

34. The method of claim 33, wherein said wired connection is a direct wired connection connecting each electrical field sensing device to said computer.

35. The method of claim 23, wherein said assay system comprises a wireless connection that connects each electrical field sensing device to said computer.

36. The method of claim 23, wherein said computer comprises a processor and a connection to a power source.

37. The method of claim 23, wherein said assay system comprises a controller that controls the voltage applied to each electrical field sensing device.

38. The method of claim 23, wherein said data is sent to said computer via a wired connection.

39. The method of claim 23, wherein said data is sent to said computer via a wireless connection.

40. The method of claim 23, wherein said computer processes said data using a software program.

41. The method of claim 40, wherein said software program interprets said data and displays the amount of electrical activity from said mammalian neuronal cell network to a user.

42. The method of claim 23, wherein said assay plate is configured to deliver a different test agent to each of said plurality of locations.

43. The method of claim 23, wherein said mammalian neuronal cell network comprises cells selected from the group consisting of cortical neurons, hippocampal neurons, glutaminergic neurons, and glial cells.

44. The method of claim 23, wherein said mammalian neuronal cell network within each of said plurality of locations is stimulated with an electrical pulse, a stimulating agent, or magnesium-free media before said adding step (b).

45. The method of claim 44, wherein said stimulating agent comprises potassium.

46. The method of claim 44, wherein said stimulating agent comprises glutamate.

47. The method of claim 44, wherein said stimulating agent comprises cyclothiazide, coriaria lactone, or tutin.

48. The method of claim 44, wherein said electrical pulse is provided by an electrode located within each of said plurality of locations.

49. The method of claim 44, wherein said method comprises determining the level of electrical activity at each of said plurality of locations (a) before stimulating said mammalian neuronal cell network within each of said plurality of locations and (b) before said adding step, thereby determining a normal activity index for each of said plurality of locations.

50. The method of claim 49, wherein said normal activity index for each of said plurality of locations is stored via said computer.

51. The method of claim 49, wherein said method comprises determining the level of seizure like electrical activity at each of said plurality of locations (a) after stimulating said mammalian neuronal cell network within each of said plurality of locations and (b) before said adding step, thereby determining an seizure activity index for each of said plurality of locations.

52. The method of claim 51, wherein said seizure activity index for each of said plurality of locations is stored via said computer.

53. The method of claim 51, wherein said method comprises determining the level of electrical activity at each of said plurality of locations (a) after stimulating said mammalian neuronal cell network within each of said plurality of locations and (b) after said adding step, thereby determining a test activity index for each of said plurality of locations.

54. The method of claim 53, wherein said test activity index for each of said plurality of locations is stored via said computer.

55. The method of claim 53, wherein said determining step (c) of claim 23 comprises comparing, for each of said plurality of locations, said test activity index to said normal activity index and said seizure activity index, wherein a test activity index having a level equal to said normal activity index or between said normal activity index and said seizure activity index indicates that the test agent that produced such a test activity index is said agent having the ability to inhibit seizure like electrical activity.

56. The method of claim 23, wherein said method comprises determining the level of seizure like electrical activity at each of said plurality of locations (a) after stimulating said mammalian neuronal cell network within each of said plurality of locations and (b) before said adding step, thereby determining an seizure activity index for each of said plurality of locations.

57. The method of claim 56, wherein said method comprises determining the level of electrical activity at each of said plurality of locations (a) after stimulating said mammalian neuronal cell network within each of said plurality of locations and (b) after said adding step, thereby determining a test activity index for each of said plurality of locations.

58. The method of claim 57, wherein said determining step (c) of claim 23 comprises comparing, for each of said plurality of locations, said test activity index to said seizure activity index, wherein a test activity index having a level less than said seizure activity index indicates that the test agent that produced such a test activity index is said agent having the ability to inhibit seizure like electrical activity.

59. The method of claim 23, wherein said method comprises using a pipette to place cells at each of said plurality of locations, thereby providing each of said plurality of locations with said mammalian neuronal cell network.

60. The method of claim 23, wherein said assay plate is placed in an incubator to support growth or maintenance of said mammalian neuronal cell network.

61. The method of claim 23, wherein said computer instructs each electrical field sensing device when to read electrical activity.

62. The method of claim 23, wherein said method comprises, after step (c):
(d) removing each different test agent from said plurality of locations;
(e) adding a second set of test agents to said assay plate such that each of said plurality of locations comprises a different test agent from said second set; and
(f) determining whether or not the presence of a test agent in at least one of said plurality of locations inhibits seizure like electrical activity of said mammalian neuronal cell network, wherein inhibition of said seizure like electrical activity indicates that said test agent is said agent having the ability to inhibit seizure like electrical activity.

63. The method of claim 62, wherein said method comprises repeating steps (d) through (f) for a third set of test agents.

64. The method of claim 62, wherein said method comprises repeating steps (d) through (f) for two to 1000 additional sets of test agents.
